(19) United States
(12) Patent Application Publication
(10) Pub. No.: US 2025/0053712 A1
(43) Pub. Date: Feb. 13, 2025

(54) ADVANCED DATA-DRIVEN MODELING FOR PURIFICATION PROCESS IN BIOPHARMACEUTICAL MANUFACTURING

(71) Applicant: Bayer HealthCare LLC, Whippany, NJ (US)

(72) Inventors: Shreya MAITI, San Ramon, CA (US); Konstantinos SPETSIERIS, San Francisco, CA (US)

(21) Appl. No.: 18/720,910

(22) PCT Filed: Jan. 24, 2023
(86) PCT No.: PCT/US2023/011409

§ 371 Date: Jun. 17, 2024

Related U.S. Application Data
(60) Provisional application No. 63/306,971, filed on Feb. 4, 2022.

(51) Int. Cl. G06F 30/27

(52) U.S. Cl. CPC G06F 30/27

(57) ABSTRACT

An exemplary method for assessing performance of an instance of a chemical process having a series of consecutive phases includes: obtaining data related to the instance of the chemical process; and evaluating, based on the data related to the instance of the chemical process, the performance of the instance of the chemical process using a plurality of performance thresholds, wherein the plurality of performance thresholds is obtained by training a hierarchical model based on one or more historical instances of the chemical process, and wherein the hierarchical model includes: a plurality of batch-evolution models (BEMs) at a first level of a hierarchy; a plurality of batch-level models (BLMs) at a second level above the first level of the hierarchy; and an overall performance model at a third level at a third level above the second level of the hierarchy.

### CROSS-REFERENCE TO RELATED APPLICATION

The present application claims the benefit of U.S. Provisional Patent Application No. 63/306,971, filed Feb. 4, 2022, the disclosure of which is hereby incorporated by reference in its entirety.

### FIELD OF INVENTION

The present disclosure relates generally to assessing performance of a chemical process, and more specifically to using machine-learning and data modeling techniques for assessing performance of an instance of a chemical process having a series of consecutive phases.

### BACKGROUND

Purification is an important process in biopharmaceutical manufacturing that allows separation of a therapeutic protein in its active form from other impurities. A typical purification process may include several chromatography-based unit operations and each unit operation may include multiple phases.

During the operation of each chromatographic step, continuous (time-series data per parameter for each batch) may be generated by the in/online sensors installed in the chromatography skids on the production floor and batch data (e.g., one data point per parameter for each batch) may be generated by at-line/offline in-process samples, respectively. These biomanufacturing process data can be leveraged for the development of advanced data-driven models that can generate insights for process experts to support their decisions and actions.

Traditionally, control charts for each of the in/on/at/offline analyses are trended univariately (e.g., one parameter per chart) to monitor a biomanufacturing process. This results in having to review multiple charts at the same time to find any correlations among the parameters. This makes real-time early fault detection and retrospective root cause analysis time-consuming and cumbersome. Additionally, trying to find relationships among multiple attributes by simply reviewing individual charts for each parameter could be exceptionally challenging and limited in capturing all the underlying correlations. Multivariate Data Analysis (MVDA) is a methodology including advanced statistical techniques that can be used to effectively analyze large, complex, and heterogeneous datasets all at the same time. The development and deployment of such MVDA models would allow for more effective and efficient near real-time process monitoring, early fault detection and diagnosis. MVDA models can be used to monitor multiple process variables with only a few multivariate metrics, while leveraging useful process information found in the correlation structure between process variables. Thus, MVDA is a powerful methodology that can be used to assist process engineers and scientists with root cause identification for process excursions and provide numerous insights into the manufacturing operations that can in turn be leveraged to enhance overall process understanding and control.

### SUMMARY

Embodiments of the present disclosure include an application of advanced data-driven modeling to an affinity chromatography column in commercial biologics manufacturing. This includes developing a multivariate model using process parameters and in-process control parameters at the purification step and visualizing the correlations among them. Specifically, embodiments of the present disclosure can be used to: (a) present the application of a hierarchical data-driven modeling methodology for effective monitoring of a purification unit operation and its corresponding phases, (b) highlight the utility of such data-driven models, and (c) provide an overview of the key steps involved in the development of advanced data-driven models in biologics manufacturing. Although the model is developed for an affinity chromatography column, similar modeling approaches can be adopted for monitoring other types of chromatography columns, such as ion-exchange, hydrogen ion concentration and others. The fundamental concept of the data-driven modeling approach discussed herein is to find correlations and patterns in the data generated during the biomanufacturing process.

An exemplary method for assessing performance of an instance of a chemical process having a series of consecutive phases comprises: obtaining data related to the instance of the chemical process; and evaluating, based on the data related to the instance of the chemical process, the performance of the instance of the chemical process using a plurality of performance thresholds, wherein the plurality of performance thresholds is obtained by training a hierarchical model based on one or more historical instances of the chemical process, and wherein the hierarchical model comprises: a plurality of batch-evolution models (BEMs) at a first level of a hierarchy, each BEM model corresponding to one phase of the series of consecutive phases; a plurality of batch-level models (BLMs) at a second level above the first level of the hierarchy, each BLM model corresponding to one phase of the series of consecutive phases; an overall performance model at a third level at a third level above the second level of the hierarchy, the overall performance model corresponding to all the series of consecutive phases.

In some embodiments, the chemical process is a purification process for separating recombinant protein from other proteins in a cell culture using one or more chromatography columns.

In some embodiments, the series of phases comprises equilibration, loading, washing, and elution of the one or more chromatography columns.

In some embodiments, the chemical process comprises a purification process, a cell culture development process, a cell isolation process, a viral inactivation process, a manufacturing process of a pharmaceutical product, or any combination thereof.

In some embodiments, each BEM of the plurality of BEMs is trained to obtain one or more performance thresholds for evaluating in-line data related to a phase in the chemical process.

In some embodiments, the one or more performance thresholds comprise a Hotelling's T2 metric and one or more model residuals.

In some embodiments, the plurality of BEMs is trained using in-line data related to the one or more historical instances of the chemical process.

In some embodiments, the in-line data comprises time-series data obtained from one or more sensors.

In some embodiments, the in-line data is interpolated at a defined frequency.

In some embodiments, each BEM model of the plurality of BEMs is a partial least squares (PLS) model.

In some embodiments, each BLM of the plurality of BLMs is trained to obtain one or more performance thresholds for evaluating in-line data, at-line data, and off-line data related to a phase in the chemical process.

In some embodiments, the one or more performance thresholds comprise a Hotelling's T2 metric and one or more model residuals.

In some embodiments, the plurality of BLMs is trained using in-line data, at-line data, and off-line data related to the one or more historical instances of the chemical process.

In some embodiments, the at-line data and off-line data comprise protein solution (bulk) attributes, bulk thaw process attributes, column load attributes, column attributes, eluate attributes, sample measurements, or any combination thereof.

In some embodiments, each BLM model of the plurality of BLMs is a principal component analysis (PCA) model.

In some embodiments, the overall performance model is trained based on the trained BLM models on the second level.

In some embodiments, the method further comprises displaying, on a display, one or more results of the evaluated performance of the instance of the chemical process.

In some embodiments, the method further comprises updating variables of the chemical process based on the evaluated performance of the instance of the chemical process.

An exemplary system for assessing performance of an instance of a chemical process having a series of consecutive phases comprises: one or more processors; a memory; and one or more programs, wherein the one or more programs are stored in the memory and configured to be executed by the one or more processors, the one or more programs including instructions for: obtaining data related to the instance of the chemical process; and evaluating, based on the data related to the instance of the chemical process, the performance of the instance of the chemical process using a plurality of performance thresholds, wherein the plurality of performance thresholds is obtained by training a hierarchical model based on one or more historical instances of the chemical process, and wherein the hierarchical model comprises: a plurality of batch-evolution models (BEMs) at a first level of a hierarchy, each BEM model corresponding to one phase of the series of consecutive phases; a plurality of batch-level models (BLMs) at a second level above the first level of the hierarchy, each BLM model corresponding to one phase of the series of consecutive phases; an overall performance model at a third level at a third level above the second level of the hierarchy, the overall performance model corresponding to all the series of consecutive phases.

An exemplary non-transitory computer-readable storage medium stores one or more programs for assessing performance of an instance of a chemical process having a series of consecutive phases, the one or more programs comprising instructions, which when executed by one or more processors of an electronic device, cause the electronic device to: obtain data related to the instance of the chemical process; and evaluate, based on the data related to the instance of the chemical process, the performance of the instance of the chemical process using a plurality of performance thresholds, wherein the plurality of performance thresholds is obtained by training a hierarchical model based on one or more historical instances of the chemical process, and wherein the hierarchical model comprises: a plurality of batch-evolution models (BEMs) at a first level of a hierarchy, each BEM model corresponding to one phase of the series of consecutive phases; a plurality of batch-level models (BLMs) at a second level above the first level of the hierarchy, each BLM model corresponding to one phase of the series of consecutive phases; an overall performance model at a third level at a third level above the second level of the hierarchy, the overall performance model corresponding to all the series of consecutive phases.

### DESCRIPTION OF THE FIGURES

FIG. 1 depicts an exemplary hierarchical model schematic, in accordance with some embodiments. Information from all Base-level models is communicated to Top-level models through their respective matrices (Tⱼ) into a consensus matrix R. R is used to further summarize data from all Base-level models by generating Top-level scores T_{TL} and loadings P_{TL}.

FIG. 2 depicts an exemplary cross-validation protocol, in accordance with some embodiments. A dataset is partitioned into training and testing sub-sets. Models are developed for each of the cross-validation rounds (with different partitioning of the dataset). Cross-validation outcomes from all models are averaged to estimate the model's final predictive power.

FIG. 3 depicts exemplary data structures (A) and (B), in accordance with some embodiments. With respect to (A) Batch Evolution models, differently colored datasets represent data from different batches. Each dataset comprises ‘m’ number of X variables (process-related variables), ‘n’ observations (time points) and one Y variable (column volume). With respect to (B) Batch Level Model, dataset columns used for Batch Evolution models are transposed to generate the dataset for the Batch Level models. Each row represents a different batch.

$${X}_{{1}_{{time}_{1}}}$$

denotes value of first X₁ variable at time₁. Similarly,

$${X}_{{m}_{{time}_{n}}}$$

denotes the value of Xₘ variables at timeₙ.

FIG. 4 depicts an exemplary schematic representation of a purification column monitoring model structure, in accordance with some embodiments. The lowest level in the hierarchy is Batch Evolution model (BEM) which is a PLS model with only inline data. The next level up in the hierarchy are Batch Level models (BLM) for each of the phases which are PCA models with inline and at-line/offline data. Finally, the Top-level model is a comprehensive PCA model comprising inline and at-line/offline data for all phases taken together.

FIG. 5 depicts an exemplary BEM score plot depicting scores of the first principal component t[1] for X space as a function of column volume (considered as maturity variable for purification process), in accordance with some embodiments. The green dashed line denotes the mean of the data and red dashed lines correspond to the +3 standard deviations from the mean.

FIG. 6 depicts exemplary model training, in accordance with some embodiments. Batch Evolution models are shown for different phases in the purification process. Each of the plots depicts progression of the batches summarized by MVDA scores t[1] for X space with respect to column volumes of material passed through the purification column.

FIG. 7 depicts exemplary model training, in accordance with some embodiments. Batch Level models for 4 phases are shown in the purification process. Each plot represents a single phase; the boundary of the ellipse indicates a 95% confidence of data used to build the models for each of the phases. Each circle within the ellipse refers to a single batch, and shows all information of the batch summarized for that particular phase by MVDA scores.

FIG. 8 depicts exemplary model training, in accordance with some embodiments. The figure shows a Top-Level model of an affinity purification column that considers 4 phases-Equilibration, Load, Wash and Elution of the process.

FIG. 9 depicts an example of model metrics used for excursion detection and diagnosis for a single BLM, in accordance with some embodiments. Specifically shown are: (A) Hotelling's T2; (B) Model residuals are both used to identify excursions during process monitoring; and (C) Contribution charts denote the contributions of variables towards an excursion for a batch, compared to the mean of all batches.

FIG. 10 depicts a model benchmarking example, in accordance with some embodiments. Also shown are model detected excursion using MVDA metrics and identified contributing process parameters using a contribution chart. Further described are: (A) Process excursion detected for a batch through MVDA metrics “Hotelling's T2” and “Residuals” in Load Phase Batch Level model (BLM); (B) Excursion confirmed by MVDA score plot of Load phase Batch Evolution model (BEM); (C) a Contribution chart used to identify process parameters associated with the excursion-column pump flow rates were [[was]] found to have the highest contribution to this excursion; and (D) Univariate representation of pump flow rate vs. Column volume: column loading pump flow rate drastically reduced for some time during Load phase of the batch due to the pump stalling for a period of time.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown but are to be accorded the scope consistent with the Claims.

### 1. Materials and Methods

### 1.1. Purification Process

The purification process is downstream of the cell culture and isolation steps in the manufacturing process of any recombinant therapeutic protein. During purification, the recombinant protein of choice is separated from a pool of myriad proteins, DNA, metabolites, etc. and synthesized by the mammalian host cell during the cell culture and other process and product related impurities. Different chromatographic columns are used during purification of a certain protein, depending on the type of protein being purified. Ion-exchange, hydrophobic interaction and affinity chromatography are among the most widely used separation techniques implemented for protein purification. The purification process is usually segregated into several phases, such as, equilibration, load, wash, elution, and finally regeneration and storage of the purification column. The multivariate models developed for online monitoring of a therapeutic protein purification process using an affinity chromatographic column are discussed herein. This column comprises peptide ligands (with target protein binding domains) on stationary phase beads that capture target protein molecules during the “Load” phase and release the protein molecules during “Elution” phase. Non-target proteins without an affinity for column ligands flow through the column as waste material.

### 1.1.1. Equilibration

During equilibration, the purification column is equilibrated with respect to its internal pH and conductivity prior to loading a target protein. This is accomplished by flowing a buffer through the column at appropriate conditions for the protein of choice.

### 1.1.2. Load

The column is first loaded with the target protein solution. During this phase, therapeutic protein molecules with affinity to the packed beads in the column bind to the beads while the impurities flow through the column to waste since they have no affinity for peptide ligands.

### 1.1.3. Wash

Wash buffer is passed through the column to dislodge only loosely bound impurities while keeping tightly held target protein molecules bound to the stationary phase beads.

### 1.1.4. Elution

Elution buffer is passed through the column that disrupts the bonds between target protein and peptide ligands and facilitates dislodging target protein molecules from the column. Column eluate containing the target protein is collected for further processing.

### 1.2. Data and Data Sources

Data is the foundation of modeling efforts described herein. There are two categories of data that are used for the development of MVDA models for the affinity chromatography column.

### In/Online Data

The inline measurements used in the model are of the following types: (a) totalized volume of effluents from chromatography column, (b) conductivity, (c) ultraviolet absorbance (UV), (d) temperature, (e) pressure and (f) flow rate. Data from process measurements are stored in a database called PI process historian (OSIsoft). All the time-series data obtained from process sensors, such as a conductivity sensor, are stored in the PI Archive and their corresponding batch context (e.g., batch ID, individual process phase start and end timestamps, etc.) is stored in the PI Asset Framework (AF) database.

### Atline/Offline Data

Both at-line and offline data are accessible via Discoverant (BIOVIA), which is a relational database. Structured Query language (SQL) is used to retrieve data from the underlying data systems such as Manufacturing Execution System (MES), Laboratory Information Management System (LIMS) and Systems Application and Products (SAP). The types of at-line/offline data used for model development include protein solution (bulk) attributes, bulk thaw process attributes, column load attributes, column attributes, eluate attributes and sample measurements.

### 1.3. Software

The following software were used in this case study:

Modeling: Simca 14.1 (Sartorius Stedim Biotech) and Matlab 2015b (MathWorks)

Data acquisition, preprocessing, visualization, and model automation: Matlab, Python 3.6 (Python Software Foundation).

### 1.4. In/online Data Pre-processing

In/online data obtained from the PI process historian needs to be processed in a standardized manner to remove any obvious abnormalities (such as chromatogram baseline offset), while capturing data as well as align them to a standard form to facilitate batch-to-batch comparison. Data pre-processing for the purification process involves the following steps: (a) interpolation, (b) segmentation, and (c) alignment.

Inline data captured by various sensors during the progress of a batch are saved in the PI historian at uneven sampling frequencies for different process parameters. To monitor how each of the batches progresses through the purification process and compare their performances, the inline data are is interpolated for all parameters at a defined frequency. Metadata including start and end timestamps, for each of the several phases comprising a batch for the affinity column, were leveraged to segment the inline data into the corresponding phases, by extracting the continuously recorded time-series data between the start and end time points. Time-series data for each column sensor were pre-processed to ensure all batches were aligned with respect to the start time of every sub-phase in the affinity purification process.

### 1.5. Multivariate Data Analysis

Multivariate Data Analysis (MVDA) refers to statistical techniques and algorithms that are used to jointly analyze data from more than two variables. Specifically, these algorithms can be used to detect patterns and relationships in data. Some applications of these methods are clustering (detection of groupings), classification (determining group/class membership) and regression (determining relationships between inputs and continuous numerical outputs). Some of the widely used MVDA techniques are Principal Component Analysis (PCA) and Partial Least Squares Projection to Latent Structures (PLS-henceforth referred to as Partial Least Squares).

### 1.5.1. Principal Component Analysis

Principal Component Analysis (PCA) is an MVDA method than can be used to obtain an overview of the underlying data without a priori information and labeling or mapping them to a target or output value. PCA can find structures and patterns in the data by reducing the dimensionality of datasets in which collinear relationships are present. The working principle of PCA is summarizing original data by defining new, orthogonal, latent variables called principal components. These principal components (PCs) include linear combinations of original variables in the dataset. They are chosen such that the variance explained by a fixed number of PCs is maximized. The values of the original data in the new latent variable space are called scores. Given a dataset described by an n×m matrix X with n observations and m variables, T denotes an n×k matrix containing the k principal component values, called scores. The coefficients p_{jq} with j=1, . . . , m and q=1, . . . , k that determine the contribution of each individual variable Xᵢⱼ with i=1, . . . , n to the principal component are called loadings. The m×k matrix P is called loading matrix and the relationship between T, X and P is given in matrix notation by equation (2.1):

$$X = {TP}^{T} + E \qquad ( 2.1 )$$

where E denotes the residual n×m matrix. The residual contains the variance not explained by the principal components 1 through k. An in-depth introduction to PCA is available in Basilevsky, A., “Statistical factor analysis and related methods: theory and applications”, John Wiley & Sons, 2009, which is incorporated by reference. The model quality is assessed with cross-validation (see section 2.5.4) as well as external datasets, if available. To this end, the R² and Q² statistics are evaluated. The R² statistic describes the fraction of the sum of squares explained by the model, while the Q² statistic conveys information about the predictive ability of the model. A detailed derivation of both is given in Eriksson, L., Byrne, T., Johansson, E., Trygg, J. and Vikstrom, E., “Multi- and megavariate data analysis: Basic Principles and Applications” (2013): 425, which is incorporated by reference herein.

### 1.5.2. Partial Least Squares

Partial Least Square (PLS) Regression is an MVDA method aiming at determining a functional relationship between inputs and outputs. The method is further described in “The Collinearity Problem in Linear Regression: The Partial Least Squares (PLS) Approach to Generalized Inverses” by Wold S, et al., published in SIAM J. Sci. Stat. Comput. 5(3) 1984:735-743 and “PLS Regression: A Basic Tool of Chemometrics” by Wold S et al. published in Intell. Lab. Syst. 58(2) 2001:109-130, which are incorporated herein by reference. Briefly, an approach similar to PCA is taken in that regression is conducted not on the original variables available in a dataset, but on fewer, orthogonal ones, called latent variables. These are linear combinations of the original variables. In contrast to PCA, where the latent variables are chosen to maximize variance, for PLS, latent variables are determined such that they maximize the covariance between the dependent and independent variables. The following operations are conducted both in the X-Space and the Y-Space to obtain a solution to the regression problem. In the X-Space, a linear transformation is defined such that:

$$T = XW * \qquad ( 2.2 )$$

$$and$$

$$X = {TP}^{T} + E \qquad ( 2.3 )$$

with T denoting the X-Scores n×k matrix, P denoting the X-Loadings m×k matrix, W* denoting the X-Weights m×k matrix and E denoting the X-Residuals n×m matrix with k<m. In the Y-Space, a transformation is sought such that:

$$Y = {UC}^{T} + G \qquad ( 2.4 )$$

with U denoting the Y-Scores n×k matrix, C denoting the Y-Weights q×k matrix, and G denoting the Y-Residuals n×q matrix. The X-Scores are selected to minimize the X-Residuals E and to be good predictors of Y, while the Y-Scores are chosen to minimize the Y-Residuals G. Similarly, to PCA, the R² and Q² can be calculated for PLS models.

### 1.5.3. Hierarchical Modeling

Hierarchical modeling facilitates combining data from different models, either PCA, PLS or both. This is typically done to summarize information from different parts of the process that are not exactly similar but are interconnected. An application of this would be combining different phases in affinity chromatography-based purification process, such as Equilibration, Load, Wash, and Elution, all of which are executed sequentially to accomplish specific goals for each of the phases and finally output a purified product.

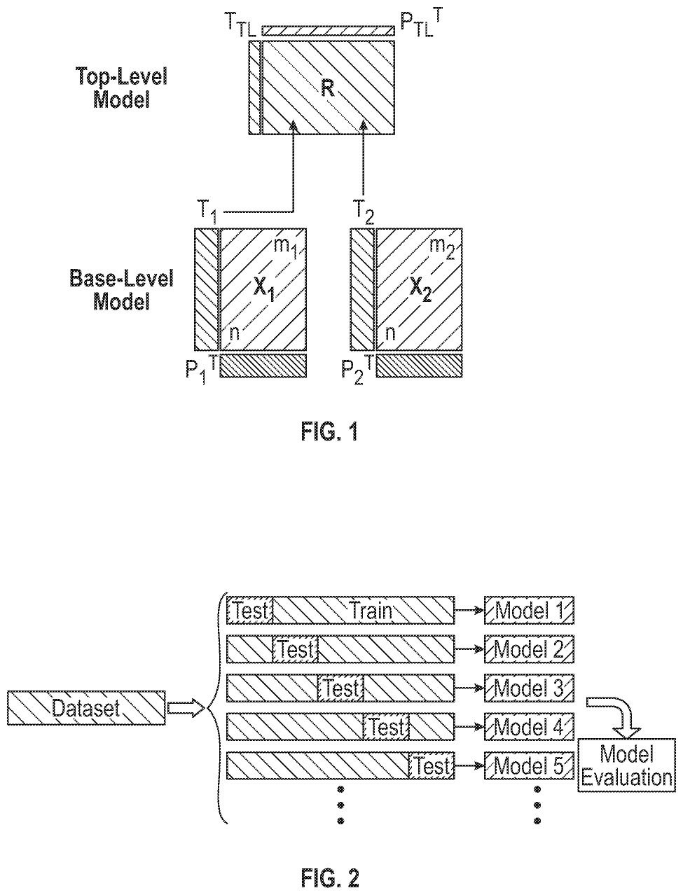

Hierarchical MVDA models comprise multiple levels. A detailed description of hierarchical models can be found in Wold, S., Kettaneh, N., Friden, H. and Holmberg, A., “Modelling and diagnostics of batch processes and analogous kinetic experiments”, Chemometrics and Intelligent Laboratory Systems 44 (1998): 331-340, which is incorporated herein by reference. An example of a two-level hierarchical model structure, with Base-level (BL) and Top-level (TL) models, is illustrated in FIG. 1 for a process with two phases 1 and 2 with data X₁ and X₂, respectively. Base-level models can (a) be multiple in number and based either on PCA or PLS, (b) summarize input data by their latent variables (i.e. score matrices Tⱼ) and (c) be described by their loading matrices, such as Pⱼ for PCA models, where j indicates the different BL models. Information from both Base-level models (corresponding to data sets X₁ and X₂) are fed into to the Top-level model through their respective score matrices T₁ and T₂ with dimensions nxk₁ and nxk₂. The number of observations are denoted by n whereas the numbers of latent variables for the BL models for phase 1 and 2 are k₁ and k₂, respectively. The TL model input is defined by the nx(k₁+k₂) matrix R comprising the scores from the two BL models. Specifically, the score matrices Tⱼ from individual X-blocks are combined to form the consensus matrix R (equation (2.5)) which is used to calculate scores and loadings for the TL model. For a PCA TL model the relationship between score matrix T_{TP}, loading P_{TP} and R matrices is given by equation (2.6), where

$$R = [ {T}_{1} , {T}_{2} ] \qquad ( 2.5 )$$

$${T}_{TP} = {RP}_{TP} \qquad ( 2.6 )$$

Generally, k_{TL}<(k₁+k₂) which indicates that the MVDA hierarchical modeling structure facilitates compressing data from all different BL models. An important benefit of hierarchical models is that each of the data blocks such as X₁, and X₂ with different dimensions retains comparable contribution to the TL model. Even if T₁ comprises fewer latent variables compared to T₂ (k₁<k₂), hierarchical modeling treats score matrices from both the BL models with similar weightage.

### 1.5.4. Cross-Validation

Cross-validation is a model testing technique used to assess if the underlying statistical relationships in the data are general enough to predict a dataset that was not used for model training. In a cross-validation technique, a given dataset is partitioned into training and testing sub-sets. A model is developed using the training dataset and then evaluated against the testing sub-set. Several rounds of cross-validation are carried out (with different partitioning), leading to multiple parallel models (see FIG. 2). The outcomes from all parallel models are averaged to estimate the final predictive power of the model. The main purpose of cross-validation is to reduce the chance of over-fitting, a condition where the model fits the training dataset very well but is not general enough to predict an independent dataset reasonably well.

### Results and Discussion

To make the MVDA purification monitoring models discussed herein a usable tool for end-users, the following factors were considered: (a) implementation of meaningful modeling approach, such that the model can detect process excursions, (b) benchmarking of new batches against historical batches. To this end, modeling work was performed in two stages: (a) model development and (b) benchmarking.

### 1.6. Model Development

The development of MVDA monitoring model for an affinity chromatography column may include three steps: model selection, model training and model testing.

### 2.1.1. Model Selection

The evaluation of batch trajectory for every single phase of the affinity chromatography, e.g., equilibration, load, wash and elution (henceforth referred to as phase) requires the development of models that account for the change in inline data as a function of batch progression. Such models are called Batch Evolution models (BEM).

Each phase can be further evaluated post-purification batch completion by considering at-line and offline data. Thus, an MVDA model is needed that can incorporate inline time-series data in addition to at-line/offline discrete process parameters and attributes. Batch Level models (BLM) can be used in this regard.

Finally, the comprehensive evaluation of the affinity chromatography unit operation requires the ability to jointly evaluate all phases. Such an objective can be achieved via a hierarchical modeling structure. The details of each of the levels in the hierarchical model are described in subsequent sections.

### 2.1.1.1 Batch Evolution Model

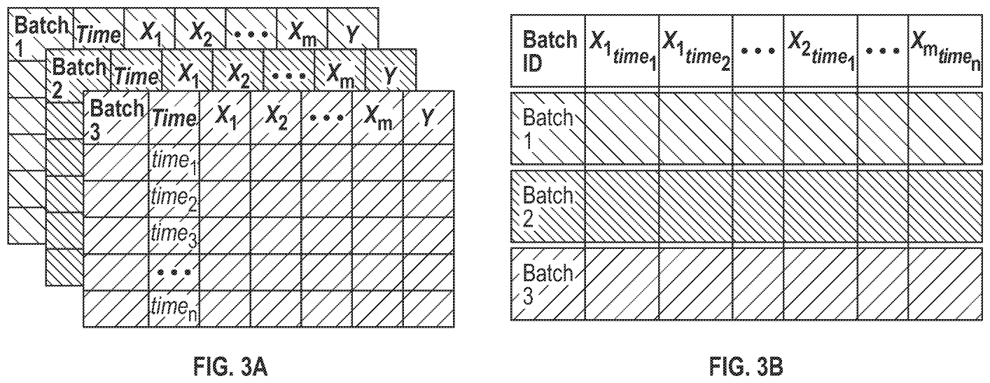

Batch Evolution model is the first level in this hierarchical model structure. Batch Evolution models provide an idea about how a batch is progressing, by considering inline data for various process parameters. The batch progression (either with respect to time of processing or volume of substance processed) is represented as a function of all available inline process parameters, which are summarized by few latent variables. BEMs are PLS models with process parameters as X variables and batch progression maturity as Y variable. In some embodiments eleven inline process parameters comprise X variables and column volume is used as variable Y. BEMs focus on maximizing covariance among all the process parameters X and batch maturity Y. The datasets used for generating BEMs comprise time-series data for multiple batches. Each of the columns in the datasets corresponds to the different variables used for model development. Each of the rows corresponds to different time points in measurement for that batch (FIG. 3A).

### 2.1.1.2 Batch Level Model

Batch Level model is the second level in the hierarchical model structure. A Batch Level model provides an idea about how a batch performed, compared to historical batches, once a phase of the purification process is completed, considering both inline and at-line/offline data. BLMs here are essentially PCA models that focus on explaining the variations present in the different process variables. All inline time-series data is transposed such that each row in a BLM dataset represents a single batch (see FIG. 3B).

### 2.1.1.3 Top-Level Model

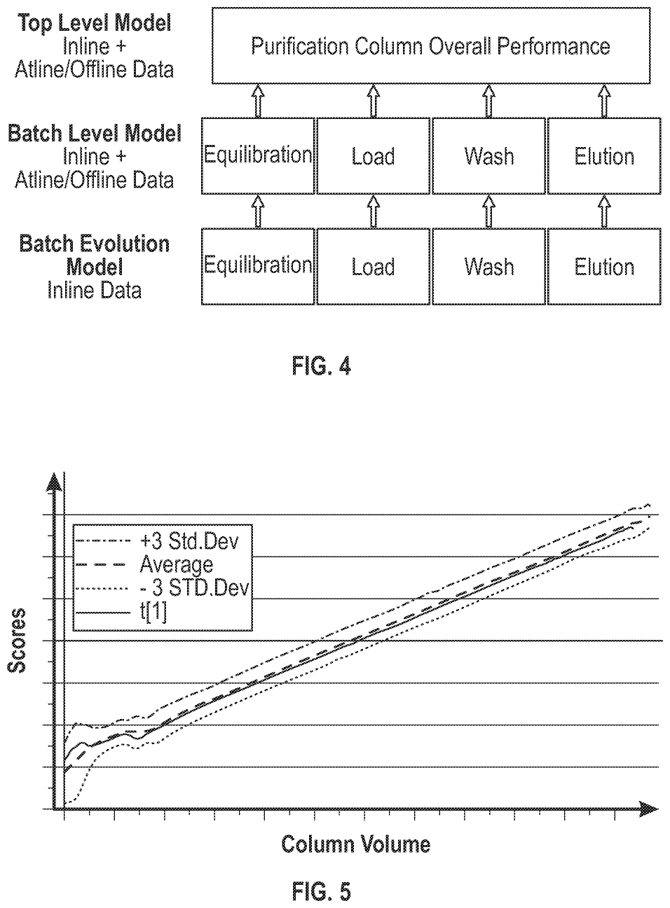

Top-level model is the third and the highest level of the hierarchical model structure. A TL model combines different levels in the multivariate modeling structure and provides a comprehensive view of the performance of a single batch through all the phases of the purification process (see FIG. 4). The lowest level in the hierarchy is a Batch Evolution model (BEM), which is a PLS model, with only inline data for each phase. The next level up in the hierarchy is a Batch Level model (BLM), which is a PCA model, combining inline and at-line/offline data for each of the phases. Finally, the Top-level model is a PCA model including inline and at-line/offline data for all phases taken together.

### 2.1.2. Model Training

After defining the structure of the model, in this case a hierarchical structure with Batch Evolution and Batch Level models at the base level and an overarching Top-level model, the next step is to train the model. Model training here refers to the process of using historical data to define multivariate control limits that in turn would be the “acceptable operating range”. Historical data comprising sixty Drug Substance (DS) batches were used for model training in some embodiments. All these batches were considered for model training, since they represent acceptable operational range. Specifically, the quality of the final product these DS batches produced was acceptable for release, hence none of the batches were eliminated for model training.

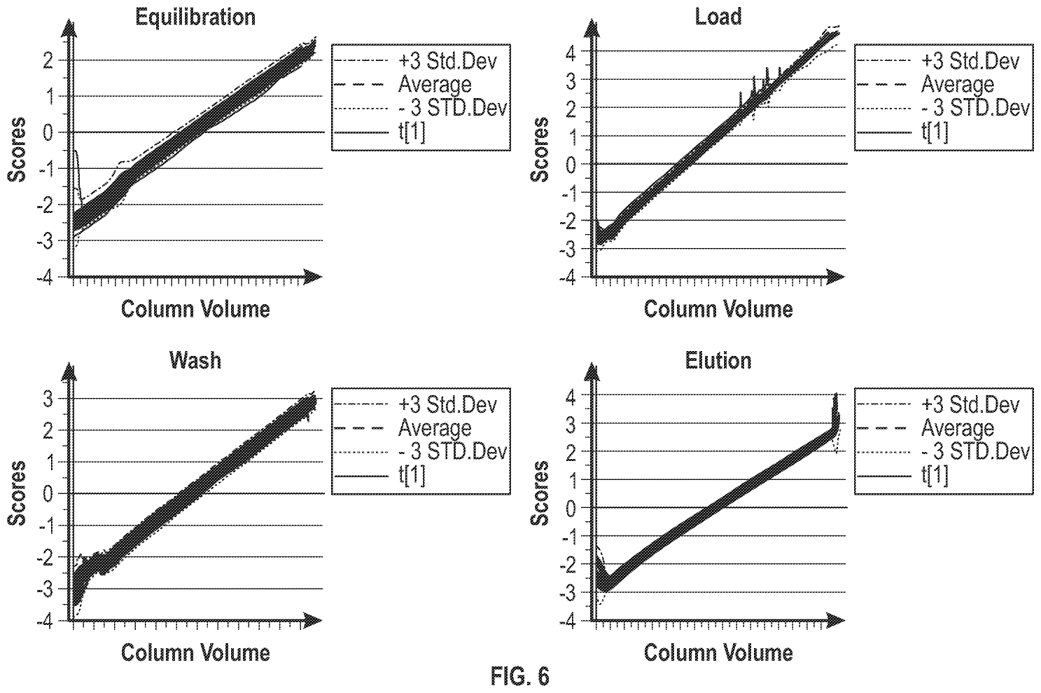

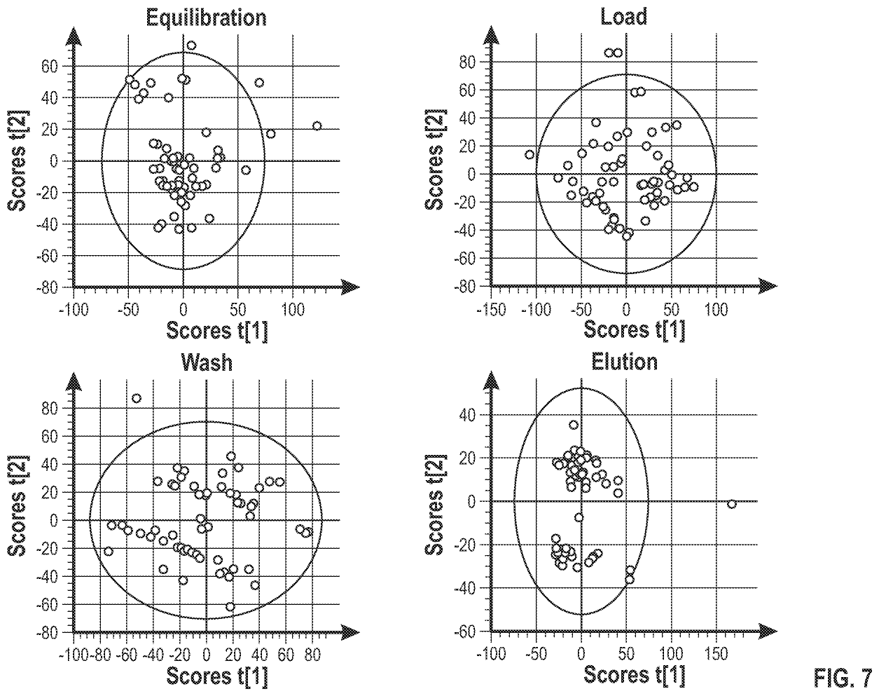

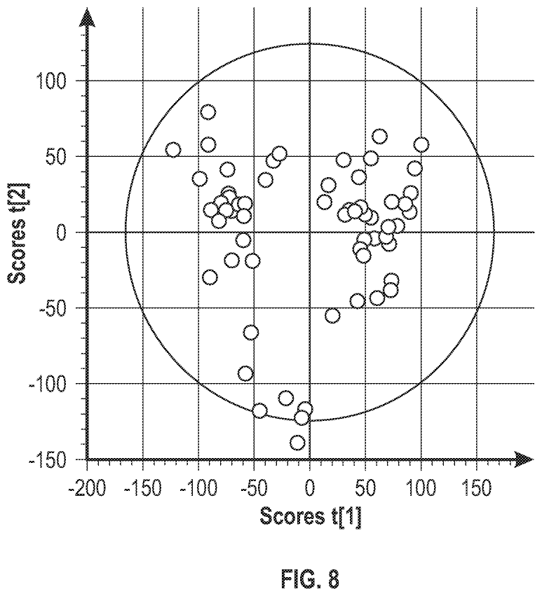

Training the model with historical data (acceptable batches) enables defining multivariate control limits that are in fact the acceptable operational ranges. At the BEM level, the original time-series data is described with few latent variables and those can be visualized as a function of column volume. In FIG. 5, the score plot of the first principal component of a single batch from BEM is shown as a function of column volume. The multivariate limits for BEMs are ±3 standard deviations (denoted by red dashed lines) of the historical data mean (shown in green dashed line). FIG. 6 shows the BEM depictions for all the purification phases and batches considered for model training. Most of the batches used for model training lie within the multivariate limits. Some batches that were outside the multivariate limit but had no process and product impact downstream of the purification process were included to increase variability in the training dataset (to reduce chances of over-fitting). Score plots for BLMs and Top-level model are shown in FIG. 7 and FIG. 8 respectively.

Process monitoring is facilitated using two multivariate metrics-Hotelling's T2 and model residuals. Hotelling's T2 represents the distance of an observation from the historical mean. Residuals refer to the part of the dataset that cannot be explained by the model, usually noise in the data or an occurrence not seen by the model before. Acceptable ranges of Hotelling's T2 and residuals for a batch is defined by the critical level of 95%. If a batch lies within the acceptable range for Hotelling's T2 and/or residuals, no action is taken. However, if a batch lies outside these acceptable ranges for either or both the metrics then further investigation of contributing factors is triggered. Contribution plots provide a quantitative comparison of potential contributions for different process parameters towards a certain excursion. It depicts the difference of a selected batch or group of batches against the mean of all batches.

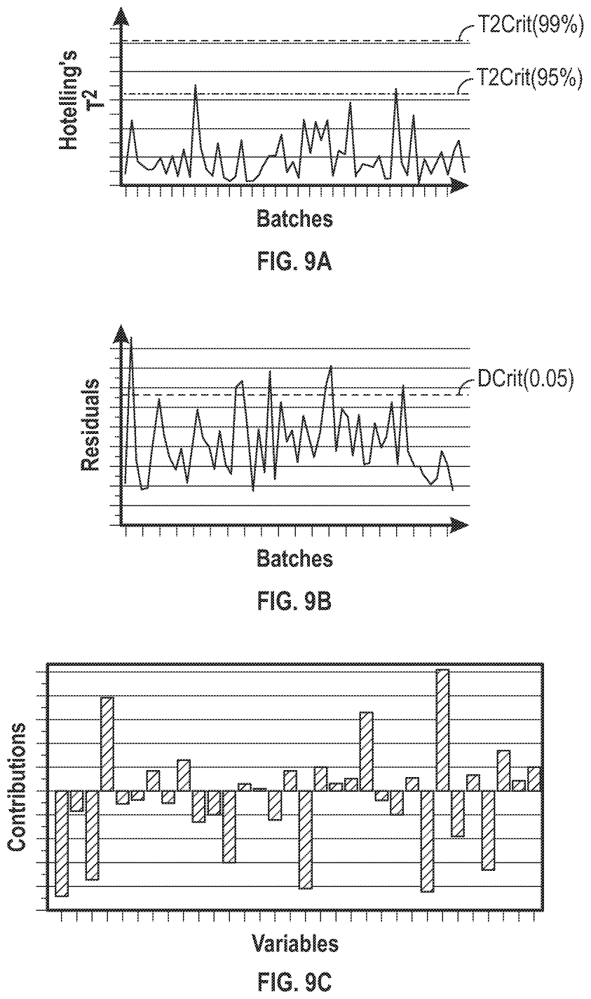

FIG. 9 shows an example of the two excursion-detection metrics (Hotelling's T2 and model residual) and one diagnostic metric (variable contribution) for a single BLM. However, these were calculated for all BLMs (shown in FIG. 7) and Top-level (shown in FIG. 8) as well.

### 2.1.3. Model Testing

MVDA models are tested based on the following objectives. First, testing is done to ensure that the models developed using a training dataset are general enough to describe an independent dataset. For this, cross-validation is implemented (see section 1.5.4). Seven rounds of cross-validation were used for model testing purpose.

Further, testing is done to demonstrate the model's ability to detect excursions and determine the underlying contributing parameters. Eleven additional batches for the affinity chromatography process have been used two-fold—to detect process excursions and for model benchmarking.

### 2.2 Model Benchmarking

Model benchmarking refers to evaluation of new batches (batches that are not used for model training) against a historical expectation that represents acceptable operational range for the process. This enables the assessment of potential excursions and if any, the investigation of the identified contributing factors.

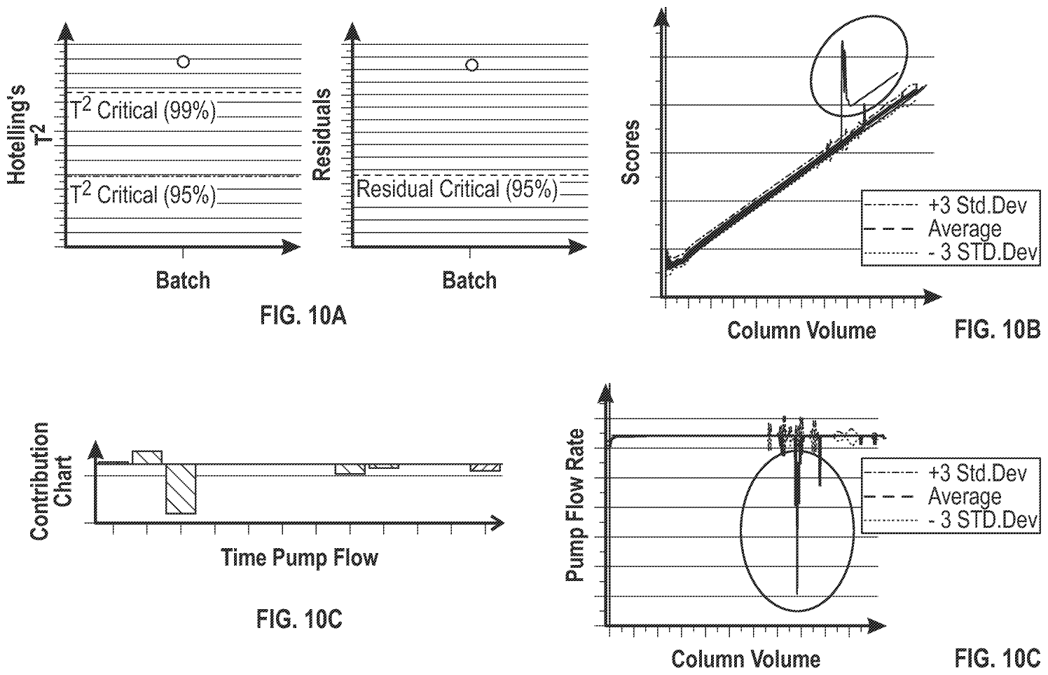

Eleven purification batches (not included in training dataset) were used for model benchmarking. This served as a test for the model's ability to detect excursions (as mentioned in Section 3.1.3 for model testing). Multivariate metrics-Hotelling's T2 and model residuals were used for evaluating batches. An example of model testing/benchmarking is shown in FIG. 10. A process excursion was detected for one of the batches in both the Hotelling's T2 and model residuals values (both being outside the acceptable levels) for the affinity chromatography column. The excursion was confirmed in the MVDA score space for the Loading Phase BEM. In the contribution plot, illustrated in FIG. 10 (C), it was noted that pump flowrate had the highest contribution to this excursion. Delving deeper into the univariate plots, it was found that the pump was stalled for some time during the Load phase FIG. 10 (D). Additionally, subject-matter experts from Manufacturing Sciences confirmed that the pump was indeed stalled due to some technical issues during the Load phase. Hence through this monitoring procedure, excursions can be detected that may or may not have an impact on product quality.

### 3. Conclusions

An abundance of process and product data are generated during commercial manufacturing of biopharmceuticals. These large and complex datasets are typically produced from in/online sensors for various unit operations as well as from benchtop analyzers on the production floor and quality control labs. This disclosure describes how the wealth of manufacturing data for a purification process can be utilized to develop advanced data-driven models that can in turn be leveraged to generate insights for process experts and support organizational decisions. Specifically, a case study was presented for preparative affinity chromatography used in the manufacture of a recombinant therapeutic protein.

Multivariate models were developed for an affinity chromatography column for the purpose of effective and efficient in/online process monitoring using available inline, online, at-line and offline data. A multivariate hierarchical modeling approach was employed to account for the several purification phases comprising the affinity chromatography unit operation and facilitate their comprehensive assessment. This implies that the hierarchical model can monitor the trajectory of process parameters for every single process phase in addition to a joint evaluation of the process parameters with the in-process controls. Specifically, individual Batch Evolution and Batch Level models were developed for each phase, enabling the evaluation of the progression of a new batch in the context of historical expectation. Available historical data were leveraged for the training of these models and additional data was used for model testing and benchmarking. The developed models describe historically accepted operating conditions which are used for evaluation of new batches. Benchmarking can be performed via few multivariate diagnostics and contribution analysis which highlight factors (original variables) potentially contributing to excursions, if any. The models presented herein were tested and shown to be capable of detecting excursions.

The current case study demonstrates how the development of advanced hierarchical data-driven models enables the effective purification process monitoring via a comprehensive assessment of all phases comprising a unit operation and the ability to detect patterns and relationships within each phase and across the different phases. The multivariate modeling also ensures efficient process monitoring, since many process parameters can be evaluated via only a few multivariate metrics while retaining the ability to drill down to the individual univariate analyses. Moreover, the modeling approach discussed herein can be applied to multiple unit operations during the biomanufacturing processes, not limited to purification alone. Developing multivariate models for the cell culture, viral inactivation, and final product manufacturing (fill and finish) processes can also provide additional process understanding and an efficient method of holistic process monitoring and early fault detection.

Overall, advanced multivariate data-driven modeling can enhance process monitoring for early fault detection and fault diagnosis for purification unit operations, while simultaneously support overall organizational efforts for process understanding and control of the biologics manufacturing process.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described to best explain the principles of the techniques and their practical applications. Others skilled in the art are thereby enabled to best utilize the techniques and various embodiments with various modifications as are suited to the particular use contemplated.

1. A method for assessing performance of an instance of a chemical process having a series of consecutive phases, comprising:
obtaining data related to the instance of the chemical process; and
evaluating, based on the data related to the instance of the chemical process, the performance of the instance of the chemical process using a plurality of performance thresholds,
wherein the plurality of performance thresholds is obtained by training a hierarchical model based on one or more historical instances of the chemical process, and
wherein the hierarchical model comprises:
a plurality of batch-evolution models (BEMs) at a first level of a hierarchy, each BEM model corresponding to one phase of the series of consecutive phases;
a plurality of batch-level models (BLMs) at a second level above the first level of the hierarchy, each BLM model corresponding to one phase of the series of consecutive phases;
an overall performance model at a third level at a third level above the second level of the hierarchy, the overall performance model corresponding to all of the series of consecutive phases.

2. The method of claim 1, wherein the chemical process is a purification process for separating recombinant protein from other proteins in a cell culture using one or more chromatography columns.

3. The method of claim 2, wherein the series of phases comprises: equilibration, loading, washing, and elution of the one or more chromatography columns.

4. The method of claim 1, wherein the chemical process comprises:
a purification process,
a cell culture development process,
a cell isolation process,
a viral inactivation process,
a manufacturing process of a pharmaceutical product, or
any combination thereof.

5. The method of any of claim 1, wherein each BEM of the plurality of BEMs is trained to obtain one or more performance thresholds for evaluating in-line data related to a phase in the chemical process.

6. The method of claim 5, wherein the one or more performance thresholds comprise a Hotelling's T2 metric and one or more model residuals.

7. The method of any of claim 1, wherein the plurality of BEMs is trained using in-line data related to the one or more historical instances of the chemical process.

8. The method of claim 7, wherein the in-line data comprises time-series data obtained from one or more sensors.

9. The method of claim 7, wherein the in-line data is interpolated at a defined frequency.

10. The method of any of claim 1, wherein each BEM model of the plurality of BEMs is a partial least squares (PLS) model.

11. The method of any of claims claim 1, wherein each BLM of the plurality of BLMs is trained to obtain one or more performance thresholds for evaluating in-line data, at-line data, and off-line data related to a phase in the chemical process.

12. The method of claim 11, wherein the one or more performance thresholds comprise a Hotelling's T2 metric and one or more model residuals.

13. The method of any of claim 1, wherein the plurality of BLMs is trained using in-line data, at-line data, and off-line data related to the one or more historical instances of the chemical process.

14. The method of claim 13, wherein the at-line data and off-line data comprise proteinsolution (bulk) attributes, bulk thaw process attributes, column load attributes, column attributes, eluate attributes, sample measurements, or any combination thereof.

15. The method of any of claim 1, wherein each BLM model of the plurality of BLMs is a principal component analysis (PCA) model.

16. The method of any of claim 1, wherein the overall performance model is trained based on the trained BLM models on the second level.

17. The method of any of claim 1, further comprising: displaying, on a display, one or more results of the evaluated performance of the instance of the chemical process.

18. The method of any of claim 1, further comprising: updating variables of the chemical process based on the evaluated performance of the instance of the chemical process.

19. A system for assessing performance of an instance of a chemical process having a series of consecutive phases, comprising:
one or more processors; a
memory; and
one or more programs, wherein the one or more programs are stored in the memory and configured to be executed by the one or more processors, the one or more programs including instructions for:
obtaining data related to the instance of the chemical process; and
evaluating, based on the data related to the instance of the chemical process, the performance of the instance of the chemical process using a plurality of performance thresholds,
wherein the plurality of performance thresholds is obtained by training a hierarchical model based on one or more historical instances of the chemical process, and
wherein the hierarchical model comprises:
a plurality of batch-evolution models (BEMs) at a first level of a hierarchy, each BEM model corresponding to one phase of the series of consecutive phases;
a plurality of batch-level models (BLMs) at a second level above the first level of the hierarchy, each BLM model corresponding to one phase of the series of consecutive phases;
an overall performance model at a third level at a third level above the second level of the hierarchy, the overall performance model corresponding to all of the series of consecutive phases.

20. A non-transitory computer-readable storage medium storing one or more programs for assessing performance of an instance of a chemical process having a series of consecutive phases, the one or more programs comprising instructions, which when executed by one or more processors of an electronic device, cause the electronic device to:
obtain data related to the instance of the chemical process; and
evaluate, based on the data related to the instance of the chemical process, the performance of the instance of the chemical process using a plurality of performance thresholds,
wherein the plurality of performance thresholds is obtained by training a hierarchical model based on one or more historical instances of the chemical process, and
wherein the hierarchical model comprises:
a plurality of batch-evolution models (BEMs) at a first level of a hierarchy, each BEM model corresponding to one phase of the series of consecutive phases;
a plurality of batch-level models (BLMs) at a second level above the first level of the hierarchy, each BLM model corresponding to one phase of the series of consecutive phases;
an overall performance model at a third level at a third level above the second level of the hierarchy, the overall performance model corresponding to all of the series of consecutive phases.
